(19) United States
(12) Patent Application Publication
(10) Pub. No.: US 2008/0281346 A1
(43) Pub. Date: Nov. 13, 2008

(54) EXPANDABLE SUPPORT DEVICE

(75) Inventors: E. Skott Greenhalgh, Lower Gwynedd, PA (US); John-Paul Romano, Chalfont, PA (US)

Correspondence Address: LEVINE BAGADE HAN LLP, 2483 EAST BAYSHORE ROAD, SUITE 100, PALO ALTO, CA, 94303, US

(73) Assignee: Stout Medical Group, L.P., Perkasie, PA (US)

(21) Appl. No.: 12/139,406

(22) Filed: Jun. 13, 2008

Related U.S. Application Data
(63) Continuation of application No. PCT/US2006/062339, filed on Dec. 19, 2006.
(60) Provisional application No. 60/752,185, filed on Dec. 19, 2005.

(51) Int. Cl. A61M 29/00; A61F 2/44

(52) U.S. Cl. 606/191; 623/17.11

(57) ABSTRACT

A device for providing support for biological tissue is disclosed. The device can expand and be implanted in lieu of removed or otherwise missing bone, such as a vertebra, and/or soft tissue, such as a intervertebral disc. The device can be configured to radially expand in a single plane when the device is longitudinally contracted. Methods for using the device are also disclosed.

### CROSS-REFERENCE TO RELATED APPLICATION

This application is a continuation of PCT International Application No. PCT/US2006/062339, filed Dec. 19, 2006 which claims the benefit of U.S. Provisional Application No. 60/752,185, filed Dec. 19, 2005, which are both incorporated herein by reference in their entireties.

### BACKGROUND OF THE INVENTION

This invention relates to devices for providing support for biological tissue, for example to repair spinal compression fractures, and methods of using the same.

Vertebroplasty is a therapy used to strengthen a broken vertebra that has been weakened by disease, such as osteoporosis or cancer. Vertebroplasty is often used to treat compression fractures, such as those caused by osteoporosis, cancer, or stress. Vertebroplasty is also often performed as an image-guided, minimally invasive procedure.

Vertebroplasty is often performed on patients too elderly or frail to tolerate open spinal surgery, or with bones too weak for surgical spinal repair. Patients with vertebral damage due to a malignant tumor may sometimes benefit from vertebroplasty. The procedure can also be used in younger patients whose osteoporosis is caused by long-term steroid treatment or a metabolic disorder.

Vertebroplasty can increase the patient's functional abilities, allow a return to the previous level of activity, and prevent further vertebral collapse. Vertebroplasty attempts to also alleviate the pain caused by a compression fracture.

Vertebroplasty is often accomplished by injecting an orthopedic cement mixture through a needle into the fractured bone. The cement mixture can leak from the bone, potentially entering a dangerous location such as the spinal canal. The cement mixture, which is naturally viscous, is difficult to inject through small diameter needles, and thus many practitioners choose to “thin out” the cement mixture to improve cement injection, which ultimately exacerbates the leakage problems. The flow of the cement liquid also naturally follows the path of least resistance once it enters the bone—naturally along the cracks formed during the compression fracture. This further exacerbates the leakage.

The mixture also fills or substantially fills the cavity of the compression fracture and is limited to certain chemical composition, thereby limiting the amount of otherwise beneficial compounds that can be added to the fracture zone to improve healing. Further, a balloon must first be inserted in the compression fracture and the vertebra must be expanded before the cement is injected into the newly formed space.

A vertebroplasty device and method that eliminates or reduces the risks and complexity of the existing art is desired. A vertebroplasty device and method that is not based on injecting a liquid directly into the compression fracture zone is desired.

### BRIEF DESCRIPTION OF THE DRAWINGS

FIG. 1 is a top view of a variation of the expandable support device in a radially contracted configuration.

FIG. 2 is a front view of the variation of the expandable support device of FIG. 1.

FIG. 3 is a top perspective view of the variation of the expandable support device of FIG. 1.

FIG. 4 is a top view of a variation of the expandable support device in a radially expanded configuration.

FIG. 5 is a front view of a variation of the expandable support device of FIG. 4.

FIG. 6 is a top perspective view of a variation of the expandable support device of FIG. 4.

FIG. 7 illustrates variations of methods for deploying the expandable support device to the vertebral column.

FIGS. 8 and 9 are top cut away views of a variation of a method for deploying the expandable support device to the vertebral column.

FIG. 10 is a top cut away view of a variation of a method for deploying the expandable support device to the vertebral column.

### DETAILED DESCRIPTION

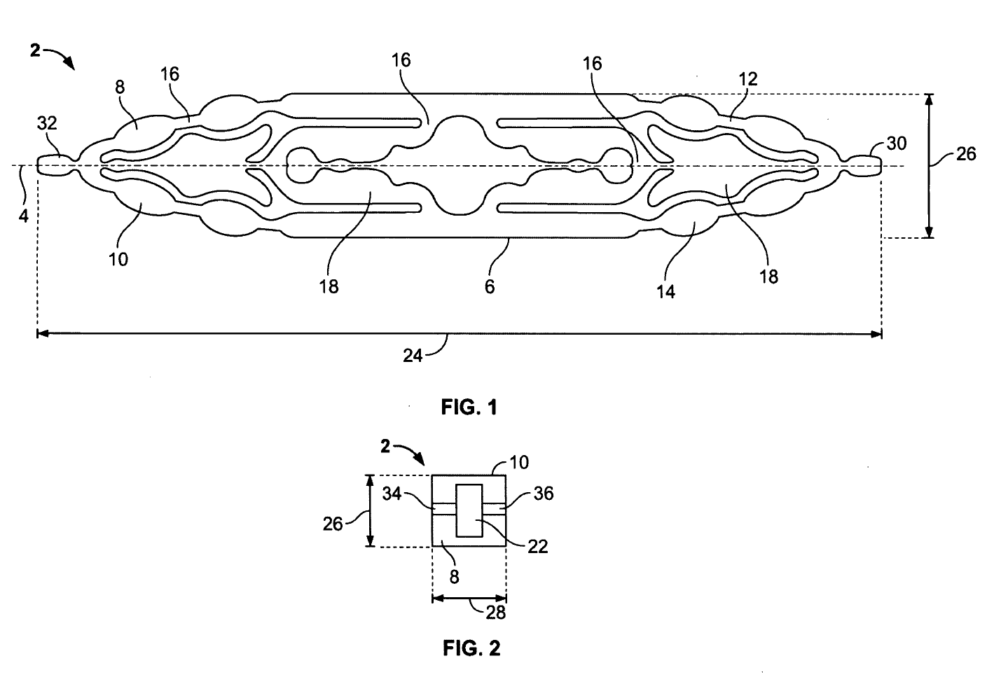

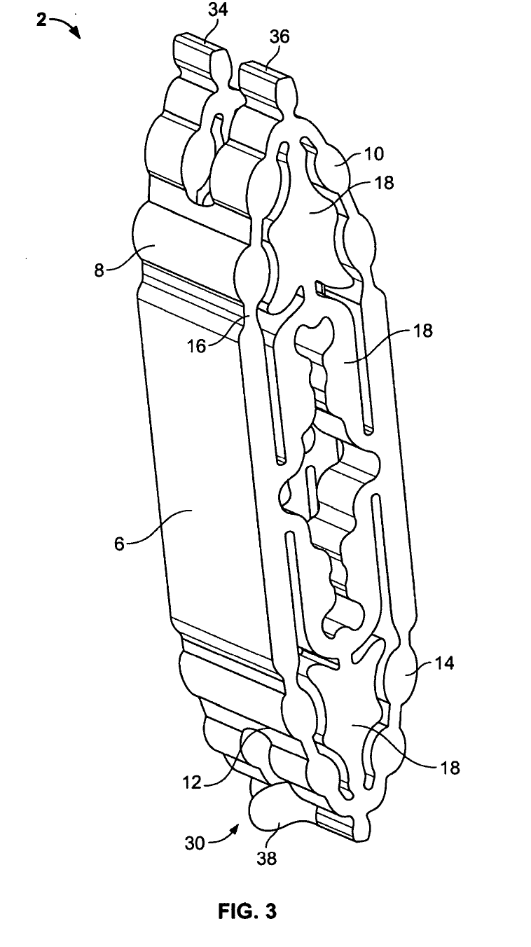

FIGS. 1 through 3 illustrate a biocompatible implant that can be used for tissue repair, for example for repair bone fractures such as spinal compression fractures, and/or repairing soft tissue damage, such as herniated vertebral discs. The implant can be an expandable support device 2. The expandable support device 2 can have a longitudinal axis 4. The expandable support device 2 can be in a first, contracted configuration, for example a radially contracted configuration. The expandable support device 2 can have a second, radially expanded configuration.

The expandable support device 2 can have one, two or more plates, such as side 6, proximal and distal plates. The side 6, proximal and distal plates can be split into multiple plates, for example a proximal first plate 8 and a proximal second plate 10, also for example, a distal first plate 12 and a distal second plate 14. The distal first plate 12 can be rotationally connected to the distal second plate 14. The proximal first plate 8 can be rotationally connected to the proximal second plate 10.

In the first configuration, the side plates 6 can be substantially parallel with the longitudinal axis 4 f the expandable support device 2. The side plates 6 can have two joints 16 in each side plate 6. The side plate 6 can be substantially rigid or flexible.

The two side plates 6 can be on opposite sides of the expandable support device 2. The distal plates can be opposite of the proximal plates.

The expandable support device 2 can have struts 18. The struts 18 can be substantially rigid. Each strut 18 can terminate with a joint 16 at one or both ends of the strut 18.

The struts 18 can be attached to each other and/or the plates at the joints 16. The joints 16 can have rotatable hinges. The hinges can be weakened portions in or near the joints 16 (e.g., in the plates and/or struts 18). For example, the hinges can be thinned portions of the plates or struts 18. The hinges can be resiliently or deformably rotatable.

The struts 18, for example at the joints 16, can be configured to be deformable and/or resilient. The struts 18 can be substantially undeformable and substantially inflexible. Each strut 18 can be flexibly (e.g., deformably rotatably) attached to one or more other struts 18 or plates. The strut 18 in the first configuration can be configured to rotate, with respect to the longitudinal axis 4 and/or the previous location of the strut 18, into the second configuration.

The expandable support device 2 can have one or more static struts 20. The static struts 20 can be configured to not rotate from the first configuration into the second configuration.

The expandable support device 2 can have a longitudinal channel 22. The longitudinal channel 22 can be substantially open or closed when the expandable support device 2 is in a radially contracted configuration.

The plates and/or struts 18 can have a thickness from about 0.25 mm (0.098 in.) to about 5 mm (0.2 in.), for example about 1 mm (0.04 in.). The longitudinal channel 22 can have an inner diameter from about 1 mm (0.04 in.) to about 30 mm (1.2 in.), for example about 6 mm (0.2 in.). The inner configuration of the longitudinal channel 22 can be square, rectangular, round, oval, triangular, or combinations thereof. The thickness and/or the inner diameter can be constant or vary with respect to the length along the longitudinal axis 4. The wall thickness and/or the inner diameter can vary with respect to the angle formed with a plane parallel to the longitudinal axis 4.

The expandable support device 2 can have a device length 24, a device width 26, and a device depth 28. In the first configuration, the device length 24 can be from about 20 mm (0.79 in.) to about 60 mm (2.4 in.), more narrowly from about 30 mm (1.2 in.) to about 35 mm (1.4 in.), for example about 35 mm (1.4 in.). In the first configuration, the device width 26 can be from about 2 mm (0.08 in.) to about 15 mm (0.59 in.), more narrowly from about 5 mm (0.2 in.) to about 8 mm (0.3 in.). The device depth 28 can be from about 2 mm (0.08 in.) to about 15 mm (0.59 in.), more narrowly from about 5 mm (0.2 in.) to about 8 mm (0.3 in.).

A position of a first strut in the first configuration can be rotated with respect to the first strut in the second configuration.

The first strut position in the first configuration can be not substantially rotated with respect to the first strut in the second configuration. The struts 18 can be dynamic or static struts 20. A first static strut in the first configuration can be configured to not be substantially rotated with respect to the first static strut in the second configuration.

The expandable support device 2 can have a substantially and/or completely hollow longitudinal port or channel 22 along the longitudinal axis 4. The longitudinal channel 22 can be filled before and/or during and/or after deployment with an agent or other material described herein or combinations thereof.

The parts of the side plates 6 can be configured to expand radially away from the longitudinal axis 4, for example in two opposite radial directions.

The expandable support device 2 can have a distal engager 30 and/or a proximal engager 32. The engagers can be configured to attach to an engagement tool. The engagement tool can be configured to deliver a compressive force along the longitudinal axis 4, for example, via the engagers. The distal 30 and/or proximal 32 engagers can be sharpened. The engagers can be split by the longitudinal channel 22. For example, the distal engager 30 can have a distal first engager and a distal second engager, and/or the proximal engager 32 can have a proximal first engager 34 and a proximal second engager 36.

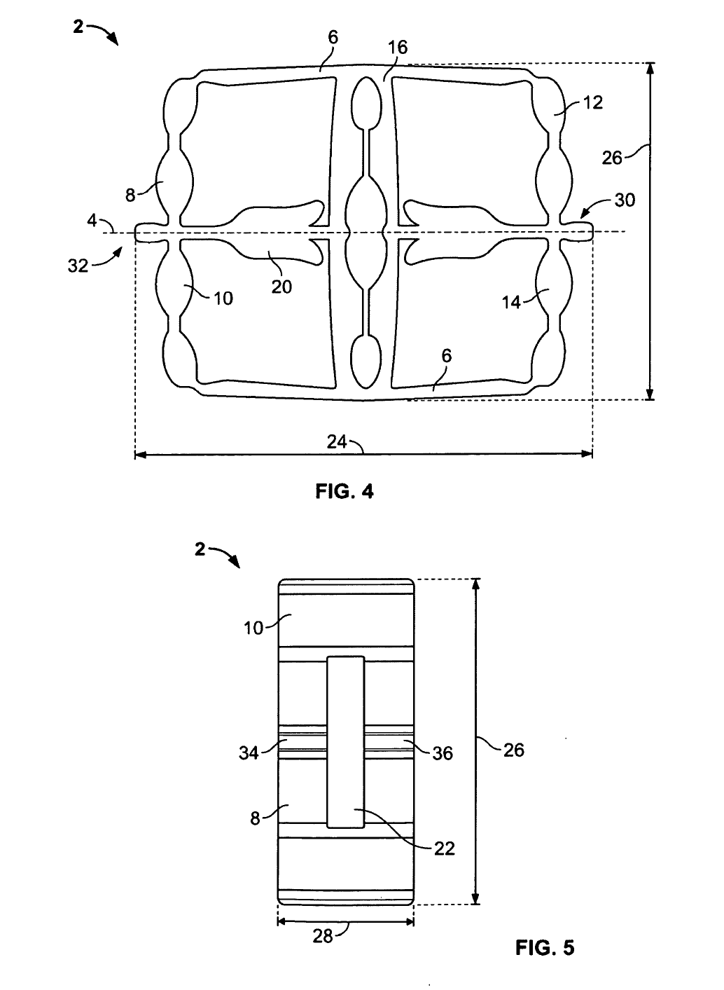

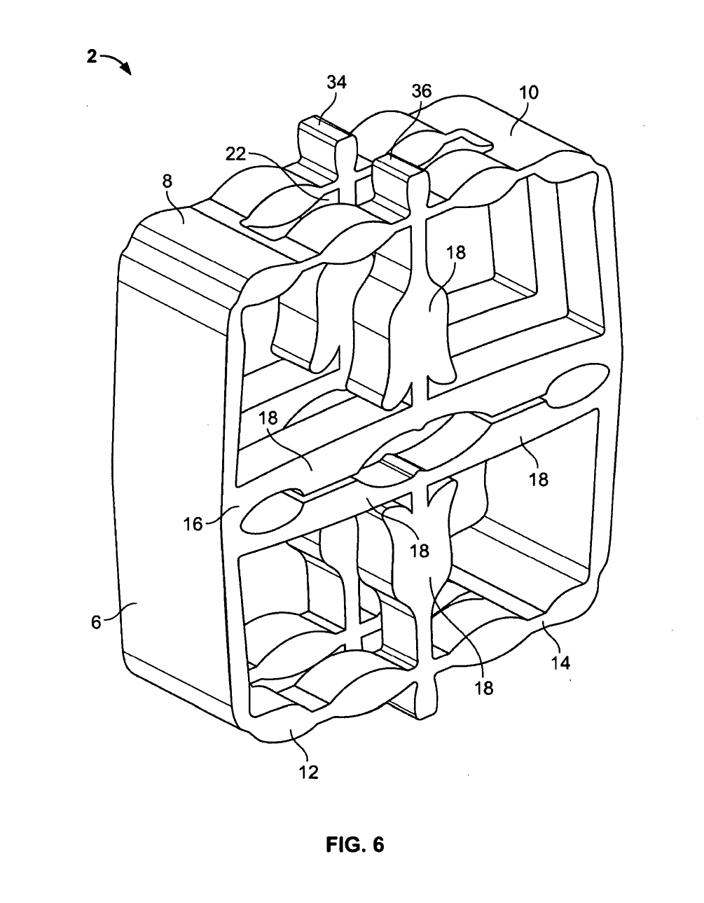

FIGS. 4 through 6 illustrate the expandable support device 2 of FIGS. 1 through 3 in a second, radially expanded configuration. The device length 24 of the second configuration can be equal to or smaller than the device length 24 of first configuration.

The radially expanded configuration can be substantially square or rectangular as seen from above (e.g., FIG. 4). In the second configuration, the distal first plate 12 can be substantially planar with the distal second plate 14. In the second configuration, the proximal first plate 8 can be substantially planar with the proximal second plate 10. The distal plates can be substantially co-planar (e.g., parallel) with the proximal plates. The expandable support device 2 can expand radially (i.e., away from the longitudinal axis 4) to change from the first configuration to the second configuration.

The distal plates can form an angle with each side plate 6, for example from about 45° to about 135°, for example about 90°. The proximal plates can form an angle with each side plate 6, for example from about 45° to about 135°, for example about 90°

In the second configuration, the device length 24 can be from about 20 mm (0.79 in.) to about 60 mm (2.4 in.), more narrowly from about 30 mm (1.2 in.) to about 35 mm (1.4 in.), for example about 30 mm (1.2 in.). In the second configuration, the device width 26 can be from about 20 mm (0.79 in.) to about 60 mm (2.4 in.), more narrowly from about 30 mm (1.2 in.) to about 35 mm (1.4 in.), for example about 30 mm (1.2 in.).

The device width 26 when the expandable support device 2 is in the first configuration can be about equal to or substantially less than the device width 26 when the expandable support device 2 is in the second configuration.

The device length 24 when the expandable support device 2 is in the first configuration can be about equal to or substantially more than the device length 24 when the expandable support device 2 is in the second configuration.

The device depth 28 when the expandable support device 2 is in the first configuration can be about equal to the device depth 28 when the expandable support device 2 is in the second configuration.

The longitudinal port or channel 22 can be configured to receive and/or slidably and fixedly or releasably attach to a locking pin (not shown). The locking pin can be used to lock the expandable support device 2 in the second configuration, for example, during or after deployment. The locking pin can be removed to remove, and/or reposition, and/or re-expand the expandable support device 2.

The expandable support device 2 can be transformed from the first configuration to the second configuration, for example, by applying a compressive force along the longitudinal axis 4 of the device and/or by applying a tensile force along an axis perpendicular to the longitudinal axis 4 and passing through the side plates 6. The expandable support device 2 can concurrently longitudinally contract and radially expand. The process can be reversed (e.g., longitudinal tension, and/or radial compression can force the expandable support device to longitudinally expand and/or radially contract.

The expandable support device 2 shown in FIG. 3 can have a mount 38, for example, for holding the expandable support device 2 vertically off a surface, for example, for presentation or accessibility purposes.

Any or all elements of the expandable support device 2 and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published Oct. 9, 2003, which is herein incorporated by reference in its entirety), tungsten-rhenium alloys, for example, as disclosed in International Pub. No. WO 03/082363, polymers such as polyethylene teraphathalate (PET), polyester (e.g., DACRON® from E. I. Du Pont de Nemours and Company, Wilmington, DE), poly ester amide (PEA), polypropylene, aromatic polyesters, such as liquid crystal polymers (e.g., Vectran, from Kuraray Co., Ltd., Tokyo, Japan), ultra high molecular weight polyethylene (i.e., extended chain, high-modulus or high-performance polyethylene) fiber and/or yarn (e.g., SPECTRA® Fiber and SPECTRAL Guard, from Honeywell International, Inc., Morris Township, N.J., or DYNEEMA® from Royal DSM N.V., Heerlen, the Netherlands), polytetrafluoroethylene (PTFE), expanded PTFE (ePTFE), polyether ketone (PEK), polyether ether ketone (PEEK), poly ether ketone ketone (PEKK) (also poly aryl ether ketone ketone), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), poly-L-glycolic acid (PLGA), polylactic acid (PLA), poly-L-lactic acid (PLLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof Examples of radiopaque materials are barium sulfate, zinc oxide, titanium, stainless steel, nickel-titanium alloys, tantalum and gold.

Any or all elements of the expandable support device 2 and/or other devices or apparatuses described herein, can be, have, and/or be completely or partially coated with agents and/or a matrix a matrix for cell ingrowth or used with a fabric, for example a covering (not shown) that acts as a matrix for cell ingrowth. The matrix and/or fabric can be, for example, polyester (e.g., DACRON® from E. I. Du Pont de Nemours and Company, Wilmington, Del.), poly ester amide (PEA), polypropylene, PTFE, ePTFE, nylon, extruded collagen, silicone, any other material disclosed herein, or combinations thereof.

The expandable support device 2 and/or elements of the expandable support device 2 and/or other devices or apparatuses described herein and/or the fabric can be filled, coated, layered and/or otherwise made with and/or from cements, fillers, glues, and/or an agent delivery matrix known to one having ordinary skill in the art and/or a therapeutic and/or diagnostic agent. Any of these cements and/or fillers and/or glues can be osteogenic and osteoinductive growth factors.

Examples of such cements and/or fillers includes bone chips, demineralized bone matrix (DBM), calcium sulfate, coralline hydroxyapatite, biocoral, tricalcium phosphate, calcium phosphate, polymethyl methacrylate (PMMA), biodegradable ceramics, bioactive glasses, hyaluronic acid, lactoferrin, bone morphogenic proteins (BMPs) such as recombinant human bone morphogenetic proteins (rhBMPs), other materials described herein, or combinations thereof.

The agents within these matrices can include any agent disclosed herein or combinations thereof, including radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

### Methods of Use

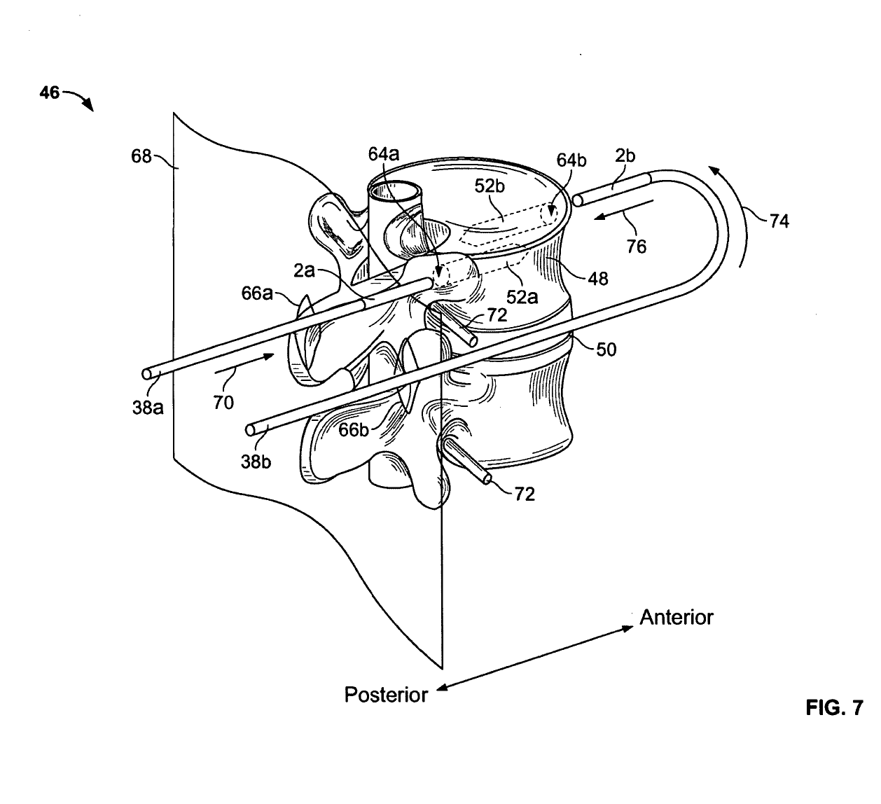

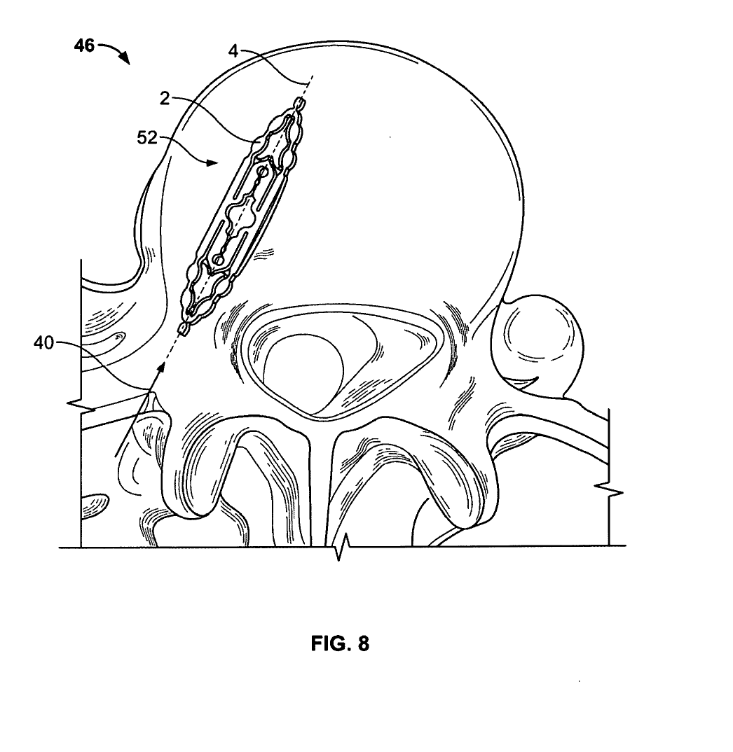

FIG. 7 illustrates that a first deployment tool 38a can enter through the subject's back. The first deployment tool 38a can enter through a first incision 66a in skin 68 on the posterior side of the subject near the vertebral column 46. The first deployment tool 38a can be translated, as shown by arrow 70, to position a first expandable support device 2a into a first damage site 52a. The first access port 64a can be on the posterior side of the vertebra 48.

With or without having an incision, the expandable support device 2 can be driven through the tissue (i.e., including the skin, if desired). For example, the distal engager 30 can cut tissue, for example with a sharpened edge.

A second deployment tool 38b can enter through a second incision 66b (as shown) in the skin 68 on the posterior or the first incision 66a. The second deployment tool 38b can be translated through muscle (not shown), around nerves 72, and anterior of the vertebral column 46. The second deployment tool 38b can be steerable. The second deployment tool 38b can be steered, as shown by arrow 74, to align the distal tip of the second expandable support device 2b with a second access port 64b on a second damage site 52b. The second access port 64b can face anteriorly. The second deployment tool 38b can translate, as shown by arrow 76, to position the second expandable support device 2 in the second damage site 52b.

The vertebra 48 can have multiple damage sites 52 and expandable support devices 2 deployed therein. The expandable support devices 2 can be deployed from the anterior, posterior, both lateral, superior, inferior, any angle, or combinations of the directions thereof.

The expandable support devices can be deployed in a vertebra, and/or between vertebra, and/or as a replacement for a vertebra.

The deployment tool can be a pair of wedges, an expandable jack, other expansion tools, any other deployment tool described in the applications incorporated by reference, or combinations thereof.

FIG. 8 illustrates that the expandable support device 2 in a first, radially contracted configuration can be longitudinally translated, as shown by arrow 40, to a treatment site 52.

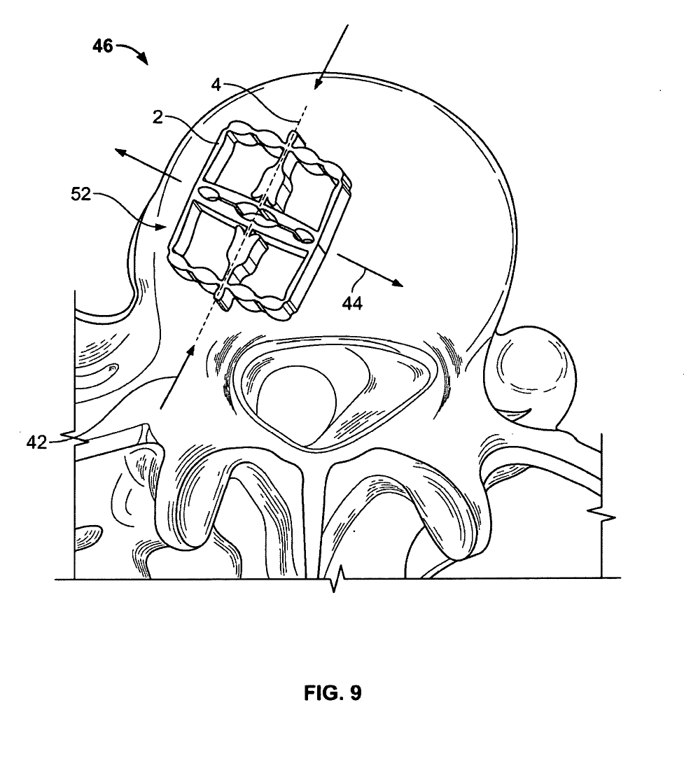

FIG. 9 illustrates that the expandable support device 2, inserted in the treatment site, can be radially expanded. A longitudinal compression, shown by arrows 42, can be aligned with the longitudinal axis 4. The longitudinal compression can cause a radial expansion, as shown by arrows 44. When deployed between vertebra, the inserted and radially expanded expandable support device 2 can have the struts 18 in contact with the adjacent vertebra and/or intervertebral discs. A locking rod or key can be inserted (not shown) into the longitudinal channel 22, for example, after radial expansion of the expandable support device 2.

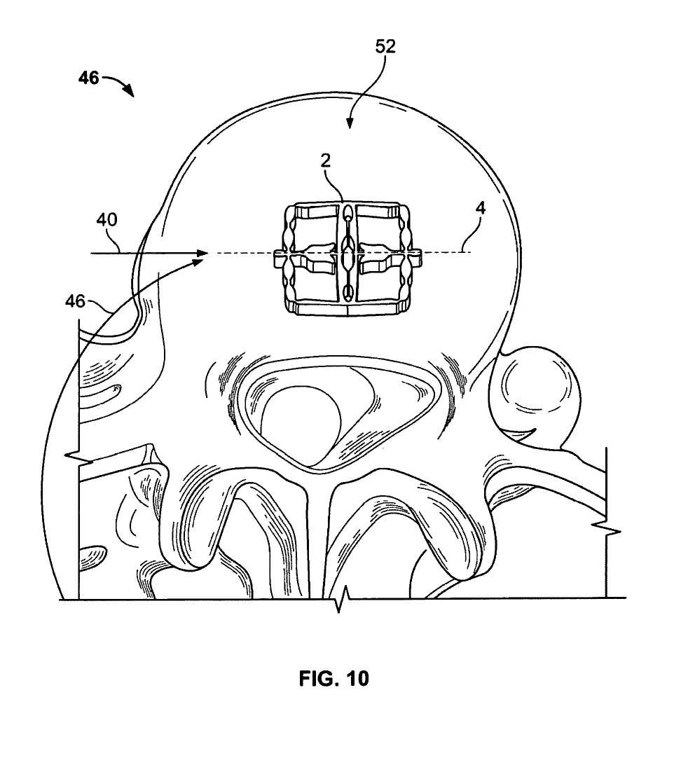

FIG. 10 illustrates that the expandable support device 2 can be inserted in the treatment site by longitudinal translation and/or longitudinal translation and rotation 46.

Additional variations of the expandable support device 2 and methods for use of the expandable support device, as well as devices for deploying the expandable support device 2 can include those disclosed in the following applications which are all incorporated herein in their entireties: PCT Application No. PCT/US2005/034115, filed Sep. 21, 2005; U.S. Provisional Patent Application No. 60/675,543, filed Apr. 27, 2005; PCT Application No. PCT/US2005/034742, filed Sep. 26, 2005; PCT Application No. PCT/US2005/034728, filed Sep. 26, 2005; PCT Application No. PCT/US2005/037126, filed Oct. 12, 2005; U.S. Provisional Patent Application No. 60/723,309, filed Oct. 4, 2005; U.S. Provisional Patent Application No. 60/675,512, filed Apr. 27, 2005; U.S. Provisional Patent Application No. 60/699,577, filed Jul. 14, 2005; and U.S. Provisional Patent Application No. 60/699,576, filed Jul. 14, 2005.

It is apparent to one skilled in the-art that various changes and modifications can be made to this disclosure, and equivalents employed, without departing from the spirit and scope of the invention. Elements shown with any variation are exemplary for the specific variation and can be in used on or in combination with other variations within this disclosure.

We claim:

1. An expandable device for orthopedic support comprising:
an expandable frame comprising a first strut and a second strut, wherein the expandable frame has a first and a second configuration;
wherein the first configuration is substantially linear; and
wherein the second configuration is substantially rectangular.

2. The device of claim 1, wherein the rectangular configuration is substantially square.

3. The device of claim 1, wherein the first strut and the second strut join at a joint and form a strut angle at the joint, and wherein the joint angle is approximately a right angle in the second configuration.

4. The device of claim 1, further comprising a longitudinal channel.

5. The device of claim 1, wherein the device has a device width, and wherein when the device is in the first configuration the device width is substantially less than the device width when the device is in the second configuration.

6. The device of claim 1, wherein the device has a device length, and wherein when the device is in the first configuration the device length is substantially more than the device width when the device is in the second configuration.

7. The device of claim 1, wherein the device comprises joints.

8. The device of claim 7, wherein the joint comprises a hinge.

9. The device of claim 8, wherein the hinge comprise a weakened portion of the strut.

10. The device of claim 7, wherein the hinge comprises a thinned portion of the strut.

11. The device of claim 1, wherein the first strut is in a first position when the device is in the first configuration, and wherein the first strut is in a second position when the device is in the second configuration, and wherein the first position is rotated with respect to the second position in a first rotation.

12. The device of claim 11, wherein the second strut is in a third position when the device is in the first configuration, and wherein the second strut is in a fourth position when the device is in the second configuration, and wherein the third position is rotated with respect to the fourth position in a second rotation, and wherein the second rotation is substantially opposite to the first rotation.

13. A method of treatment for an orthopedic damage site with an expandable support device having a longitudinal axis, the method comprising:
inserting the expandable support device through a soft tissue, wherein the expandable support device cuts the soft tissue;
deploying the expandable support device in the damage site, wherein deploying comprises converting the expandable support device from a first configuration to a second configuration; and wherein deploying comprises compressing the expandable support device along the longitudinal axis.

14. The method of claim 13, wherein the inserting comprises inserting along a linear path.

15. The method of claim 13, wherein the inserting comprises inserting along a radial path.

16. The method of claim 13, wherein deploying comprises applying tension along an axis substantially perpendicular to the longitudinal axis.

17. The method of claim 13, wherein the expandable support device in the second configuration is widened relative to the expandable support device in the first configuration.

18. The method of claim 17, wherein the expandable support device in the first configuration is longitudinally expanded relative to the expandable support device in the second configuration.

19. The method of claim 17, wherein the expandable support device in the second configuration is not substantially deepened relative to the expandable support device in the first configuration.

20. The method of claim 19, wherein the expandable support device widens along a plane substantially co-planar to a near surface of the bone.
